(19) United States
(12) Patent Application Publication
(10) Pub. No.: US 2022/0111121 A1
(43) Pub. Date: Apr. 14, 2022

(54) TISSUE ENGINEERING BONE SCAFFOLD AND PREPARATION METHOD THEREOF

(71) Applicant: Chinese PLA General Hospital, Beijing (CN)

(72) Inventors: Quanyi GUO, Beijing (CN); Xujiang LUO, Beijing (CN); Shibi LU, Beijing (CN); Shuyun LIU, Beijing (CN); Xiang SUI, Beijing (CN); Jingxiang HUANG, Beijing (CN); Gang HAN, Beijing (CN)

(21) Appl. No.: 17/269,231

(22) PCT Filed: Jan. 7, 2020
(86) PCT No.: PCT/CN2020/070708

§ 371 Date: Feb. 17, 2021

(30) Foreign Application Priority Data
Jun. 28, 2019 (CN) 201910572711.9

(51) Int. Cl. A61L 27/36; A61L 27/56; A61L 27/54; A61K 38/18

(52) U.S. Cl. CPC A61L 27/3687; A61L 27/3691; A61L 27/3608; A61L 27/56; A61L 2430/02; A61K 38/1875; A61K 38/18; A61L 2300/414; A61L 27/54

(57) ABSTRACT

The present application relates to a tissue engineering bone scaffold and a preparation method thereof, and the method includes the following steps: a cleaning step, subjecting tissue engineering bone material to supercritical fluid cleaning treatment to remove soft tissue in the bone material and obtain an initial bone matrix; a sterilization step, sterilizing the initial bone matrix by supercritical fluid to obtain a bone matrix; and a compounding step, compounding a cytokine into pores of the bone matrix by means of supercritical fluid to obtain the bone scaffold.

### CROSS REFERENCE TO RELATED APPLICATIONS

The present application is a national phase of PCT application No. PCT/CN2020/070708 filed on Jan. 7, 2020 and claiming the priority of the Chinese patent application 201910572711.9, titled by “TISSUE ENGINEERING BONE SCAFFOLD AND PREPARATION METHOD THEREOF” and filed on Jun. 28, 2019, both of which are entirely incorporated herein by reference.

### TECHNICAL FIELD

The present application relates to a technical field of medical materials, and particularly relates to a tissue engineering bone scaffold and a preparation method thereof.

### BACKGROUND

Bone defect caused by etiology like trauma, infection, tumor excision, or congenital disease is increasing day by day, and in most cases, it is difficult for the bone defect to heal by itself, and repair and treatment of the bone defect have always been one of the clinical problems.

Tissue engineering is a comprehensive application of engineering and life science principles and technologies, in which a biologically active implant is pre-built in vitro and then implanted into the body to achieve purpose of repairing tissue defects and rebuilding tissue functions. The research results of the tissue engineering have promoted development of bone tissue engineering, providing new technical means for the treatment of the bone defect. In order to achieve a final application of the tissue engineering bone in the treatment of clinical bone defect, the tissue engineering bone is required to have good mechanical property and biocompatibility.

### SUMMARY

A first aspect of the present application provides a method for preparing tissue engineering bone scaffold, the method includes the following steps:
a cleaning step, subjecting tissue engineering bone material to supercritical fluid cleaning treatment to remove soft tissue in the bone material and obtain an initial bone matrix;
a sterilization step, sterilizing the initial bone matrix by supercritical fluid to obtain a bone matrix; and
a compounding step, compounding a cytokine into pores of the bone matrix by means of supercritical fluid to obtain the bone scaffold.

A second aspect of the present application provides a tissue engineering bone scaffold prepared by the method according to the first aspect of the present application, wherein the bone scaffold includes a bone matrix with a porous structure and a cytokine loaded into the pores of the bone matrix.

### BRIEF DESCRIPTION OF THE DRAWINGS

In order to more clearly explain the technical solutions of the embodiments of the present application, the accompanying drawings required in the embodiments of the present application will be briefly described below. Obviously, the accompanying drawings described below are only some embodiments of the present application. For the person skilled in the art, without paying any creative work, other drawings may be obtained based on the accompanying drawings.

FIG. 1 shows images of bone material in Embodiment 1 before supercritical fluid cleaning (A) and after supercritical fluid cleaning (B).

FIG. 2 shows hematoxylin-eosin staining (shortly referred as HE) images of bone matrix after subcutaneous implantation for one, two, and four weeks.

FIG. 3 shows crystal violet staining images in an in vitro cell migration experiment.

FIG. 4 is an X-ray film of the supercritical cleaned and sterilized bone matrix prepared according to Embodiment 1 after implantation into the bone defect area of the rabbit radius for 1 month.

FIG. 5 is an X-ray film of a bone scaffold prepared according to Embodiment 3 after implantation into the bone defect area of the rabbit radius for 1 month.

### DETAILED DESCRIPTION

In order to make technical purposes, technical solutions and beneficial technical effects of the present application clearer, the present application will be described in detail below combining with specific embodiments. It shall be understood that the embodiments described in the specification are only for explaining the present application, not for limiting the present application.

For simplicity, only a few numerical ranges are explicitly disclosed. However, any lower limit may be combined with any upper limit to define an unspecified range; and any lower limit may be combined with any other lower limit to define an unspecified range, and likewise any upper limit may be combined with any other upper limit to define an unspecified range. Further, although not explicitly stated, each point or single numerical value between end points of a range is included in the range. Thus, each point or single numerical value may serve as a lower limit or an upper limit itself, and may be combined with any other point or single numerical value, or other lower or upper limits, to define an unspecified range.

In the description herein, it should be noted that, unless otherwise stated, “above” and “below” a value means including the value, and the word “more” in the expression “one or more” means two or more.

The above summary of the present application is not intended to describe each disclosed embodiment or each implementation of the present application. The following description more specifically exemplifies exemplary embodiments. In many places throughout the application, guidance is provided by means of a series of embodiments, which may be used in various combinations. In various embodiments, a list is only a representative group and should not be interpreted as an exhaustive list.

Embodiments of the present application propose a preparation method for a tissue engineering bone scaffold. The method includes a cleaning step S100, a sterilization step S200, and a compounding step S300.

In S100, a tissue engineering bone material is subjected to supercritical fluid cleaning treatment to remove a soft tissue in a bone material to obtain an initial bone matrix.

In S200, the initial bone matrix is sterilized by supercritical fluid to obtain bone matrix.

In S300, a cytokine is compounded into pores of the bone matrix by supercritical fluid to obtain a bone scaffold.

The preparation method of the tissue engineering bone scaffold provided by the embodiment of the present application uses the supercritical fluid to clean the tissue engineering bone material to obtain the initial bone matrix, and then uses the supercritical fluid to sterilize the initial bone matrix, which can effectively reduce immunogenicity in the tissue engineering bone material and can kill pathogenic microorganism carried by the tissue engineering bone material, so that the tissue engineering bone scaffold has a relatively high biocompatibility and the immune rejection of the host to the implanted bone scaffold may be reduced.

Then, the cytokine is compounded into the pores of the bone matrix by the supercritical fluid. The cleaning, sterilization and compounding treatments by means of supercritical fluid significantly reduce damage to bone collagen and bone matrix of the tissue engineering bone material, maintain an integrity of biological macromolecule, do not damage the three-dimensional porous structure of the bone material and can provide an excellent microenvironment required by cell growth, thereby benefiting cell growth, proliferation and redifferentiation, and promoting repair of the bone defect. The supercritical fluid treatment also can preserve mechanical property of the bone material, so that the tissue engineering bone scaffold has good biomechanical property, can better maintain mechanical stability of the bone scaffold after implanted and provide mechanical support for a longer period of time, and thus is suitable for repair and reconstruction of segmental bone defect and bone nonunion.

Further, the supercritical fluid has a strong ability to dissolve and diffuse the cytokine; by the supercritical fluid, the cytokine can be carried deeply into the bone matrix, so as to be evenly distributed in the bone matrix; thus, beneficial factors, such as increasing of release time of the cytokine, reducing of a distance between the cytokine and functional action area, and availability of time-space release of the cytokine within the bone scaffold, can be achieved, so that the cytokine may be locally and slowly released to achieve local regulation of osteogenic and osteoclastic activity. By using such bone scaffold, multiple signal pathways in bone regeneration can be activated at right place and right time to achieve better bone tissue regeneration, thereby prompting integration of the implanted bone scaffold with the host bone and regeneration and repair of the bone defect, and meanwhile, reducing dosage and side effects of the cytokine.

By the preparation method of the tissue engineering bone scaffold provided in the embodiments of the present application, the obtained tissue engineering bone scaffold exhibits high osteoinductive activity, significantly promotes early osteogenesis and late osseointegration, makes the newly formed bone tissue have excellent morphology, structure, mechanical property and physiological function, and has excellent application potential.

According to the preparation method of the tissue engineering bone scaffold provided by the embodiments of the present application, the obtained tissue engineering bone scaffold may be degraded and absorbed, the degradation products have no toxic and side effects on the body, and the degradation rate matches the rate of new bone formation.

The tissue engineering bone material may be one or more of allogeneic cancellous bone, xenogeneic cancellous bone and autologous cancellous bone. The allogeneic cancellous bone has similar biomechanical property and structure to autologous bone, has characteristics of a wide range of sources, etc., and is a good bone defect repair material that may be used to replace the autologous bone. The individual who the xenogeneic cancellous bone is obtained from and the individual whose bone defect is to be repaired, do not belong to the same species. For example, the individual whose bone defect is to be repaired is a human, while the individual who the xenogeneic cancellous bone is obtained from, may be an animal, such as a pig, a cow and a sheep.

The cytokine may include one or more of transforming growth factor (transforming growth factor-β, referred to as TGF-β for short), transforming growth factor-β family (TGF-β family) and vascular endothelial growth factor (referred to as VEGF for short). In the transforming growth factor-β family, BMP-2, BMP-4, BMP-6, and BMP-7 are currently found to have osteogenic property. BMP-2 belongs to the TGF-β family, and it is secreted by osteoblast and acts on the osteoblast. BMP-2 is an important signal molecule currently found to promote osteoblast differentiation and bone extracellular matrix synthesis and secretion, and it plays a role in inducing osteoblast differentiation, expresses in limb growth and fracture, and plays an important role in bone growth and development, and bone regeneration and repair. Bone morphogenetic protein produced by recombination is named rhBMP. At present, BMP-2 and BMP-7 have been produced by recombination and are respectively named rhBMP-2 and rhBMP-7.

According to the preparation method of the tissue engineering bone scaffold provided by the embodiment of the present application, the growth factor in the tissue engineering bone scaffold can be effectively prevented from being denatured due to a processing procedure, a sterilization procedure, or interaction with a toxic organic solvent. More preferably, the tissue engineering bone scaffold can make the cytokine gradually release at a certain concentration, which can not only achieve a local small effective drug stimulation concentration for a long time period, but also inhibit potential complications caused by overdose of the cytokine. Therefore, the use of the tissue engineering bone scaffold can provide a good therapeutic effect for the bone that needs a long time to grow and recover, and can increase the rate and quality of the bone formation, while reducing inflammation and other risks.

The supercritical fluid is a fluid between a gas and a liquid that is above a critical temperature and a critical pressure, and has both properties and advantages of gas and liquid. The supercritical fluid has strong solubility, good diffusion performance, and is easy to be controlled, and has characteristics of good stability, non-toxicity, easy separation and environmental protection. In the cleaning step S100, the sterilization step S200, and the compounding step S300, the supercritical fluid is independently selected from one or more of supercritical carbon dioxide, supercritical water, supercritical alcohol and supercritical alkane. The supercritical alcohol is, for example, supercritical methanol and supercritical ethanol. The supercritical alkane is, for example, C1 to C12 alkane in a supercritical state.

In some embodiments, the cleaning step S100 includes placing the bone material in a supercritical fluid environment to dissolve soft tissue into the supercritical fluid and thus remove the soft tissue. A pressure in the supercritical fluid environment is 9 MPa to 15 MPa. A temperature of the supercritical fluid environment is 37° C. to 45° C., preferably 38° C. to 42° C. The cleaning time may be 10 h to 20 h, such as 14 h to 18 h, and for example, 16 h.

Under the above operating conditions, the immunogenicity in the bone material can be reduced, so that the tissue engineering bone scaffold has a high biocompatibility. Further, the above operating conditions have little damage to the bone collagen, bone matrix or the like of the bone material, the integrity of the biomacromolecule is good, and the three-dimensional porous structure and mechanical property of the bone material can be well maintained.

In the cleaning step S100, a pressure relief rate after cleaning may be 0.1 MPa/min to 1 MPa/min, for example, 0.5 MPa/min.

The cleaning step S100 may be carried out in supercritical carbon dioxide equipment of Nova2200 type, which includes a carbon dioxide cylinder, a cooler, a high pressure pump, a constant temperature preheater, a controllable electric heating system, an access valve, a valve before a kettle, a reaction ball kettle, a pressure gauge, a pressure relief valve and a computer control system connected in sequence. The parameters of the equipment can be set through a main screen to control pressure and temperature inside the reaction vessel, as well as the running time and the pressure relief rate.

In some embodiments, in the cleaning step S100, the supercritical fluid may further contain a first additive, for example, hydrogen peroxide solution. The supercritical fluid added with the first additive can further improve the effect of oxidative degreasing and sterilization on the bone material, and the cleaning effect is better.

In some preferred embodiments, a mass percentage content of hydrogen peroxide in the first additive, the hydrogen peroxide solution, is 1% to 5%, for example, 2% to 4%, such as 3%.

Further, a volume ratio of the hydrogen peroxide solution to the supercritical fluid may be 1:1000 to 1:2000, for example, 1:200 to 1:1500, such as 1:1250.

In some embodiments, before the cleaning step S100, a preliminary cleaning step S400 may be included: the bone material is subjected to ultrasonic centrifugal cleaning and/or high-pressure water gun washing with a washing liquid to remove one or more of bone marrow, fat and residual blood from the bone material, wherein the washing liquid is one or more of water, alcohols and ketones.

The preliminary cleaning step S400 can effectively remove most of the bone marrow, fat and residual blood inside the bone material. In the cleaning step S100, it is easier for the supercritical fluid to enter inside of the bone tissue through the pores, which is beneficial to extract and separate the grease in the micropores, and thus can reduce the treatment time of the cleaning step S100, improve the cleaning effect, and improve the biocompatibility of the bone scaffold.

In some embodiments, before the preliminary cleaning step S400, a pretreatment step S500 may further be included. The pretreatment step S500 includes:

S510, mechanically clearing the bone material to remove the soft tissue on a surface of the bone material.

In the step S510, a cutter may be used to remove muscle, tendon, fascia and other soft tissues on the surface of the bone material.

S520, the bone material is cleaned with phosphate buffered saline (Phosphate Buffered Saline, referred to as PBS for short) solution.

PBS may be phosphate buffered saline solutions known in the art. As an example, PBS in which 8 g of NaCl, 0.2 g of KCl, 1.44 g of Na₂HPO₄, and 0.24 g of KH₂PO₄ are dissolved in 1000 mL of distilled water, may be used. The pH of PBS is, for example, 7.4.

As an example, PBS may be used to clean the bone material for 1 to 6 times, for example, 2 to 5 times, such as 3 times. Time for each cleaning may be 2 min to 10 min, for example, 3 min to 8 min, such as 5 min. The cleaning manner may be standing immersion, shaking immersion, rinsing, centrifugal cleaning, and the like.

In the step S520, the cleaning with the PBS may remove most of red blood cells in the bone material, thereby reducing the immunogenicity of the bone material and improving the biocompatibility of the bone scaffold.

S530, the cleaned bone material is frozen at −10° C. to −50° C. for 10 h to 48 h, and then is frozen at −50° C. to −100° C. for 10 h to 48 h.

In an alternative embodiment, the cleaned bone material is frozen at −20° C. for 24 h, and then is transferred to −80° C. and frozen for 24 h.

By the treatment in the step S530, the immunogenicity of the bone material can be initially reduced, efficiency of the subsequent cleaning can be improved, while the bone collagen, bone matrix or the like in the bone material are not damaged, and the biomechanical property of the bone material is preserved.

In some embodiments, before the step S520, the method further includes:

S540, cutting the mechanically cleared bone material into bone pieces with a predetermined volume.

In the step S540, a bone saw may be used to cut the bone material. The predetermined volume is, for example, (5 to 20) mm×(5 to 20) mm×(5 to 20) mm, such as 10 mm×10 mm×10 mm. There is no particular restriction on the shape of the bone piece, which may be selected according to actual requirements, such as cuboid, cube, cylinder, etc.

In some embodiments, the bone material after treated by the step S530 may be directly treated in the subsequent step, or may be placed in an environment of −20° C. and stored for later use.

In some embodiments, the sterilization step S200 includes: making the supercritical fluid to penetrate into the initial bone matrix to sterilize the initial bone matrix.

The sterilization step S200 may be performed in supercritical carbon dioxide equipment of Nova2200 type. The initial bone matrix is placed in the supercritical fluid, a pressure of the supercritical fluid may be 12 MPa to 15 MPa, and the supercritical fluid penetrates into the initial bone matrix to sterilize the initial bone matrix. A temperature of the sterilization treatment may be 37° C. to 45° C., preferably 38° C. to 42° C. The time for sterilization may be 0.5 h to 3 h, for example 0.8 h to 1.2 h, such as 1 h.

Under the above operating conditions, the immunogenicity of the bone material can be effectively reduced, and the pathogenic microorganism carried by the tissue engineering bone material can be killed, so that the tissue engineering bone scaffold has a higher biocompatibility. Further, the above operating conditions have little damage to the bone collagen, bone matrix, and the like of the bone material, the integrity of the biomacromolecule is good, and the three-dimensional porous structure and mechanical property of the bone material can be well maintained.

In the sterilization step S200, a pressure relief rate after the sterilization may be 0.1 MPa/min to 1 MPa/min, for example 0.5 MPa/min.

In some embodiments, in the sterilization step S200, the supercritical fluid may contain a second additive, and the second additive is one or more of peroxyacetic acid solution and hydrogen peroxide solution. The supercritical fluid added with the second additive may better improve the sterilization effect on the bone material.

In the peroxyacetic acid solution used for the second additive, a mass percentage content of peroxyacetic acid is, for example, 5% to 20%, and for example, 10% to 20%, such as 18%. In the hydrogen peroxide solution used for the second additive, a mass percentage content of hydrogen peroxide is 1% to 5%, for example, 2% to 4%, such as 3%.

In some preferred embodiments, the second additive is a mixed solution of the peroxyacetic acid solution and the hydrogen peroxide solution, and a volume ratio of the peroxyacetic acid solution to the hydrogen peroxide solution in the mixed solution is preferably 1:9 to 9:1, for example, 2:1 to 6:1, such as 78:22.

Further, in the sterilization step S200, a volume ratio of the peroxyacetic acid solution, the hydrogen peroxide solution and the supercritical fluid is preferably 3 to 4:1:70000 to 100000, such as 3.5 to 3.6:1:80000 to 95000.

In some embodiments, after the sterilization step S200 and before the compounding step S300, a washing step S600 may be further included: using phosphate buffered saline solution PBS to wash the sterilized bone matrix and drying the washed bone matrix. The PBS as described above may be used for the phosphate buffered saline solution PBS.

In some embodiments, the compounding step S300 includes: placing the bone matrix and the cytokine in a supercritical fluid environment, and wherein the cytokine is carried into the pores of the bone matrix by the supercritical fluid and compounded with the bone matrix to obtain the bone scaffold. Wherein, a pressure of the supercritical fluid environment may be 8 MPa to 12 MPa, such as 9.9 MPa. A temperature of the supercritical fluid environment is 37° C. to 45° C., preferably 38° C. to 42° C. The treatment time may be 0.5 h to 4 h, for example, 1 h to 3 h, such as 2 h.

Under the above operating conditions, the supercritical fluid can carry the cytokine deeply into the hone matrix, so that the cytokine is evenly distributed in the bone matrix, thereby better achieving the beneficial factors, such as increasing of the release time of the cytokine, reducing a distance between the cytokine and the functional action area, and availability of time-space release of the cytokine within the bone scaffold. The bone scaffold may locally and slowly release the cytokine to achieve local regulation of osteogenic and osteoclastic activity. By using such bone scaffold, the multiple signal pathways in bone regeneration can be activated at the right place and the right time, so as to achieve better bone tissue regeneration and better promote the integration of the implanted bone scaffold with the host bone, and the regeneration and repair of the bone defect. Further, the dosage and side effects of the cytokine are also reduced.

In the compounding step S300, a pressure relief rate after the compounding may be 0.1 MPa/min to 1 MPa/min, for example 0.5 MPa/min.

In the compounding step S300, a loading concentration or amount of the cytokine may be quantified by calculating a volume of the bone matrix.

In some embodiments, the compounding step S300 includes:

S310, in a sterile environment with a temperature lower than 25° C., loading the cytokine onto the bone matrix to obtain a mixture;

S320, placing the mixture into the supercritical fluid, and the cytokine is carried into the pores of the bone matrix by the supercritical fluid and compounded with the bone matrix to obtain a bone scaffold.

The step S320 may be carried out in supercritical carbon dioxide equipment of Nova2200 type; the mixture is packed in a Tyvek-Poly pouch for compounding, and this is beneficial for the cytokine to penetrate into the bone matrix more efficiently. After that, the supercritical fluid is changed in state to be removed, and the bone scaffold is obtained.

The prepared bone scaffold is packaged in a sterile manner, and may be placed in a sterile environment at −20° C. to 4° C. and stored for later use.

The embodiments of the present application further provide a tissue engineering bone scaffold, which is prepared by the above preparation method. The bone scaffold includes:
a bone matrix with porous structure; and
a cytokine loaded into pores of the bone matrix.

The tissue engineering bone scaffold of the embodiments of the present application has relatively high biocompatibility, and may reduce the immune rejection effect of the host on the implanted bone scaffold. Further, the bone scaffold preserves the original bone collagen and bone matrix of the tissue engineering bone material, the integrity of the biomacromolecule is good, and it also preserves the three-dimensional porous structure of the bone material, which can provide an excellent microenvironment for cell growth, thereby being conductive to cell growth, proliferation and redifferentiation, and promoting the repair of the bone defect. The bone scaffold also preserves the mechanical property of the bone material, has good biomechanical property, can better maintain the mechanical stability after implanted, can provide mechanical support for a longer period of time, and is suitable for repair and reconstruction of segmental bone defect and bone nonunion.

Further, the cytokine penetrates deeply into the bone matrix and is evenly distributed in the bone matrix, and the beneficial factors, such as increasing of the release time of the cytokine, reducing of a distance between the cytokine and the functional action area, and availability of time-space release of the cytokine within the bone scaffold can be achieved. The bone scaffold can locally and slowly release the cytokine to achieve local regulation of osteogenic and osteoclastic activity. By using such bone scaffold, the multiple signal pathways in bone regeneration can be activated at the right place and right time, so as to achieve better bone tissue regeneration, promote the integration of the implanted bone scaffold with the host bone, and the regeneration and repair of the bone defect, and further reduce the dosage and side effects of the cytokine.

The tissue engineering bone scaffold according to the embodiments of the present application exhibits high osteoinductive activity, significantly promotes early osteoanagenesis and late osseointegration, and enables newly formed bone tissue to have excellent morphology, structure, mechanical property and physiological function, and has excellent application potential.

The tissue engineering bone scaffold according to the embodiments of the present application may be degraded and absorbed, the degradation products have no toxic and side effects on the body, and the degradation rate matches the rate of new bone formation.

### EMBODIMENTS

The following embodiments describe the disclosure of the present application more specifically, and these embodiments are only for illustrative purposes, because it is obvious for the person skilled in the art to make various modifications and changes within the scope of contents disclosed in the present application. Unless otherwise stated, all portions, percentages, and ratios reported in the following embodiments are based on weight, all reagents used in the embodiments are commercially available or may be obtained by synthesizing with conventional methods, and may be directly used without further treatment, and all instruments used in the embodiments are commercially available.

In the following embodiments, a purity of supercritical carbon dioxide was 95% to 99%; the PBS solution was PBS solution obtained by dissolving 8 g of NaCl, 0.2 g of KCl, 1.44 g of Na₂HPO₄, and 0.24 g of KH₂PO₄ in 1000 mL of distilled water.

### Embodiment 1

1) Muscle, tendon, fascia and other soft tissues on a surface of a bone material of a pig femoral cancellous bone was removed, the bone material then was sawed into cube bone pieces of 10 mm×10 mm×10 mm, put into the PBS solution and cleaned 3 times, cleaning for 5 min each time; then the bone pieces were put into a cold storage of −20° C. and frozen for 24 h, and after that, the bone pieces were transferred to −80° C. and frozen for 24 h so as to destroy the enzyme activity related to osteogenesis or osteoclast; then, the bone pieces were put into a freezer of −20° C. and stored for later use, wherein the storage time may be up to more than 3 months.

2) The above-mentioned stored bone pieces were washed with deionized water under pressure to remove bone marrow, fat and blood stain in the bone pieces; then, the bone pieces were put into supercritical carbon dioxide equipment of Nova2200 type for extraction and cleaning, wherein the extraction and cleaning were performed in a supercritical carbon dioxide environment at a temperature of 40° C. and a pressure of 9.9 MPa for 16 h, the first additive was 16 mL H₂O₂ solution with a mass concentration of 3%, and a volume ratio of the H₂O₂ solution to the supercritical carbon dioxide was 1:1250.

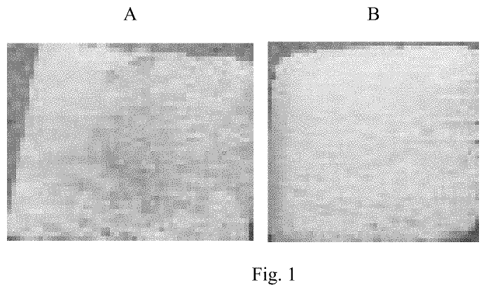

FIG. 1(A) showed an image of the bone piece after washed with deionized water under pressure, and FIG. 1(B) showed an image of the bone piece after the supercritical carbon dioxide cleaning. As can be seen from the comparison of A and B in FIG. 1, the supercritical carbon dioxide cleaning effectively removed the remaining soft tissues and cells in the bone pieces, which effectively reduced the immunogenicity of the tissue engineering bone material, so that the tissue engineering bone scaffold had high biocompatibility.

3) The cleaned bone pieces were sterilized by supercritical carbon dioxide, wherein the sterilization temperature was 40° C., the pressure was 12 MPa, the volume of the supercritical carbon dioxide was 20,000 mL, the second additive was 0.78 mL peroxyacetic acid solution with a mass concentration of 18% and 0.22 mL H₂O₂ solution with a mass concentration of 3%, and the sterilization time was 1 h. After that, the bone pieces were washed repeatedly by sucking up the PBS solution using a pipette tip of 1 mL, strictly following the sterility requirements in a sterile ultra-clean table; after washing, the bone pieces were dried with sterile gauze and the supercritically cleaned bone matrixes were obtained for later use.

Testing Method

(0.1) Compressive strength of the bone piece: each group of samples was prepared to a uniform specification (10 mm×10 mm×10 mm), and then was tested by a mechanical testing machine; a formal test was performed after pre-compression of 200N for 3 times, a test rate was 3 mm/min, and the test was ended when deformation of the bone tissue occurred; a force-displacement curve was obtained, and then a stress-strain curve was obtained by using a software, Origin8.5, and the maximum compressive strength and compressive elastic modulus of the bone pieces were obtained.

(2) Porosity of the bone piece: The porosity (Porosity) of the bone pieces was measured by the method of absolute ethyl alcohol replacement. A syringe with graduation of 10 mL was chosen and filled with a certain amount of absolute ethyl alcohol, and an initial volume V₁ of the ethyl alcohol was obtained; the bone piece was formed into a cuboid of 5 mm×5 mm×5 mm and put into the syringe and completely saturated by the ethyl alcohol, and thus a volume V2 of the ethyl alcohol after the bone piece was saturated, was obtained; the bone piece saturated with the ethyl alcohol was taken out, and a volume of the remaining ethyl alcohol was V3; three samples were measured and an average was obtained. The porosity was calculated according to the following formula:

Porosity(ε)=(V₁-V₃)/(V₂-V₃)×100%

(3) Aperture of the bone piece: the aperture of the bone piece was measured by using a scanning electron microscope of S-4800 type from Japan Hitachi (Hitachi) company, and a particle size analysis software, Nano Measurer1.2.

Table 1 showed the measurements of compressive strength and the porous structure of the bone pieces after washed with deionized water under pressure (Comparative Examples 1-1 to 1-2) and the bone pieces after supercritical carbon dioxide cleaning and sterilization (Embodiments 1-1 to 1-4), wherein the bone pieces in Embodiments 1-1 to 1-4 were taken from different pig femurs, the bone pieces in Comparative Example 1-1 and Embodiment 1-1 were taken from the same pig femur, and the bone pieces in Comparative Example 1-2 and Embodiment 1-2 are taken from the same pig femur. As can be seen from data in Table 1, the three-dimensional porous structure and mechanical property of the bone material were better maintained by means of supercritical fluid treatment to the bone material.

TABLE 1

| | Compressive | Maximum | | |
|---|---|---|---|---|
| | elastic | compressive | | |
| | modulus | strength | Porosity | Aperture |
| | ( GPa) | (GPa) | (%) | (μm) |
| Embodiment 1-1 | 0.29 | 6.26 | 75 | 270 |
| Embodiment 1-2 | 1.08 | 6.63 | 80 | 260 |
| Embodiment 1-3 | 0.54 | 8.26 | 65 | 190 |
| Embodiment 1-4 | 1.38 | 10.60 | 85 | 280 |
| Comparative | 0.30 | 6.28 | 76 | 260 |
| Example 1-1 | | | | |
| Comparative | 1.08 | 6.65 | 80 | 240 |
| Example 1-2 | | | | |

### Comparative Example 1

1) after removing soft tissues such as muscle, tendon and fascia on a hone surface of a pig femoral cancellous bone, the pig femoral cancellous bone was cut into cubic bone pieces of 10 mm×10 mm×10 mm, and then, the bone pieces were put into PBS solution and cleaned 3 times, cleaning for 5 min each time; then, the bone pieces were put into a cold storage of −20° C., frozen for 24 h, and then transferred to −80° C. and stored for 24 h to destroy the enzyme activity related to osteogenesis or osteoclast; after that, the bone pieces were put into a freezer of −20° C. and stored for later use.

2) The bone pieces obtained in step 1) were immersed in methanol/chlorofom of 1:1 at 40° C., and degreased and cleaned for 24 h; after that, the bone pieces were taken out and cleaned with methanol for 2 h.

3) The bone pieces obtained in step 2) were soaked in medical alcohol with a mass concentration of 75% for 2 h and were sterilized.

4) The bone pieces obtained in step 3) were cleaned with deionized water 5 times, cleaning for 15 minutes each time, and then were dried in a dryer at 60° C., and thus the bone matrixes were obtained. The bone matrixes were stored at −20° C. for later use; however, the bone pieces cleaned by traditional methanol/chloroform still had strong immunogenicity and moderate toxicity.

### Embodiment 2

Twelve healthy SD rats, each of which weighed about 200 g, were anesthetized and then skins of the anesthetized rats were prepared; conventional sterilized drape was used, and an incision of 1.5 cm was made in the back of each rat, subcutaneous separation; for each rat, one bone matrix subjected to the supercritical carbon dioxide cleaning and sterilization (the preparation method was described in the Embodiment 1) and one bone matrix cleaned and sterilized by conventional methanol/chloroform (preparation method was described in Comparative Example 1), in the same volume and size, were implanted subcutaneously one on each side, and the incisions were sutured by surgical suture; the rats were respectively sacrificed at 1 week, 2 weeks, 4 weeks after surgery, the sacrificed rats were fixed with 4% paraformaldehyde for 48 h, routinely dehydrated, embedded and sectioned, and the sections were made with hematoxylin-eosin staining and observed under a microscope.

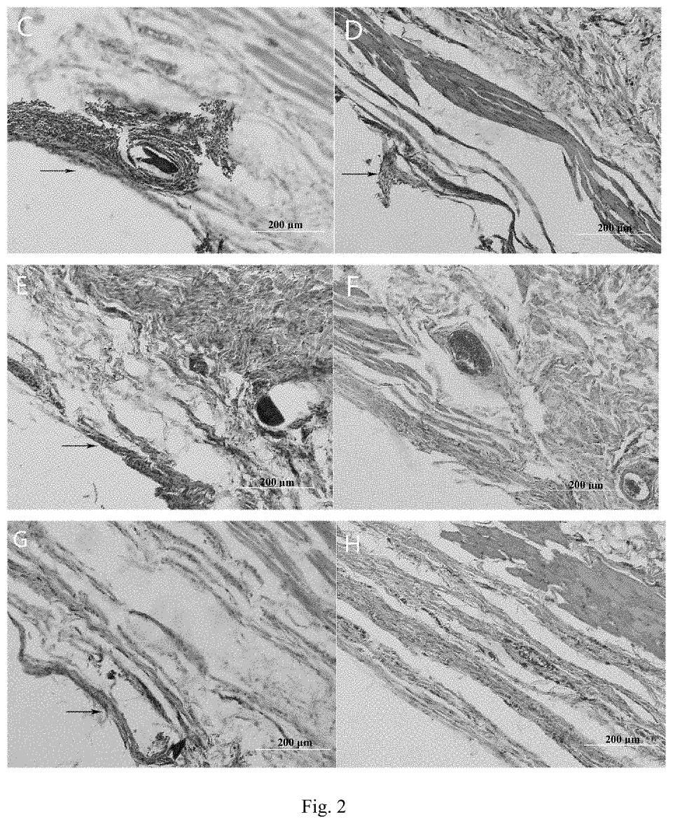

The histological analysis of the bone matrixes after implanted respectively for 1 week, 2 weeks, and 4 weeks was shown in FIG. 2, wherein C, E, and G were HE staining images of the bone matrixes cleaned and sterilized by conventional methanol/chloroform and subjected to subcutaneous embedding in the rats for 1 week, 2 weeks, and 4 weeks, and D, F, and H were HE staining images of the bone matrixes subjected to the supercritical carbon dioxide cleaning and sterilization and subcutaneous embedding in the rats for 1 week, 2 weeks and 4 weeks. As can be seen from the figures, at 1 week after surgery, both groups had dense infiltration of inflammatory cells, but the inflammatory cells of the rats implanted with the bone matrixes subjected to the supercritical carbon dioxide cleaning and sterilization were significantly reduced; at 2 weeks after surgery, the numbers of inflammatory cells in both groups significantly decreased, but the rats implanted with the bone matrixes conventionally cleaned and sterilized still had more inflammatory cells, while the rats implanted with the bone matrixes subjected to the supercritical carbon dioxide cleaning and sterilization had no obvious inflammatory cells; at 4 weeks after surgery, the number of the inflammatory cells around the bone matrixes in both groups further decreased, but there was still a small amount of inflammatory cells infiltrated around the bone matrixes conventionally cleaned and sterilized, while no obvious inflammatory cells were found around the implanted bone matrixes subjected to the supercritical carbon dioxide cleaning and sterilization. The results of the present embodiment showed that the supercritical carbon dioxide cleaning and sterilization technology reduced the inflammatory reaction of the implanted bone material, and made the tissue engineering bone scaffold have higher biocompatibility.

### Embodiment 3

1) The bone matrix obtained in the Embodiment 1 was taken out, which had a volume of 1 cm³.

2) The cytokine, recombinant human bone morphogenetic protein-2 (rhBMP-2) and/or recombinant human vascular endothelial growth factor (rhVEGF-165) was dispersed in PBS solution, and a cytokine suspension was obtained, in which a concentration of the cytokine was 50 μg/mL; the cytokine suspension was loaded according to a volume ratio V (a volume of cytokine suspension): V (a volume of bone material)=100 μL: 1 cm³, and thus an amount of rhBMP-2 and/or rhVEGF-165 loaded on the bone scaffold in the present Embodiment was 5 μg.

3) In the bone matrix loaded with the cytokine, the cytokine was transported into the pores of the bone matrix by supercritical carbon dioxide delivery technology, where the supercritical temperature was 40° C., the pressure was 9.9 MPa, and the time was 2 h, and the tissue engineering bone scaffold loaded with the cytokine was obtained. The bone scaffold was stored at −20° C.; the storage time was up to 3 months, the cytokine was still active, and the mechanical property of the bone scaffold did not significantly decrease.

### Comparative Example 2

1) After removing soft tissues such as muscle, tendon and fascia on a bone material surface of a pig femoral cancellous bone, the pig femoral cancellous bone was cut into cubic bone pieces of 10 mm×10 mm×10 mm, and the bone pieces were put into PBS solution and cleaned 3 times, cleaning for 5 min each time; then, the bone pieces were put into a cold storage of −20° C., frozen for 24 h, and then transferred to −80° C. and stored for 24 h to destroy the enzyme activity related to osteogenesis or osteoclast; after that, the bone pieces were put into a freezer of −20° C. and stored for later use.

2) The bone pieces prepared in step 1) were put into a freeze dryer and were subjected to freeze drying at −60° C. for 24 h, so that the remaining moisture in the bone tissue was reduced to a mass percentage of less than 5% and bone matrixes were obtained.

3) The bone matrixes were immersed in PBS containing rhBMP-2 at a concentration of 50 ug/mL; after the bone matrixes were saturated with the liquid, the bone pieces were put into a freeze dryer and subjected to freeze drying at −60° C. for 4 h, the remaining moisture on the surface of the hone matrixes was removed, the cell growth factor was fixed on the bone matrixes, and bone scaffolds were obtained; the bone scaffolds were stored at −20° C. for later use. The storage time was up to 3 months, the cytokine was still active, while the compression mechanical property of the bone scaffolds significantly reduced, and the bone scaffolds were fragile under pressure.

### Embodiment 4

in order to evaluate the influence of the bone tissue loaded with cytokine rhBMP-2 on the migration ability of bone marrow mesenchymal stem cells, the present embodiment used the Transwell chemotactic migration system to perform in vitro evaluation.

Two 3-4 week old New Zealand white rabbits were anesthetized, skin preparation were made, and conventional sterilized drapes were used; at the highest point of the iliac bone, the bone of each rabbit was punched and the bone marrow was extracted; after heparinization, bone marrow mesenchymal stem cells were obtained by density gradient centrifugation; the bone marrow mesenchymal stem cells were added with DMEM culture medium with serum, resuspended and placed in a CO₂ incubator of 37° C. and 5% volume fraction for culture; P3 generation cells were taken for experiment.

1×10⁴ rabbit bone marrow mesenchymal stem cells were seeded in the upper chamber of a 24-well Transwell plate (pore diameter: 8 μm) of Corning Corporation of the United States, and a bone scaffold loaded with the cytokine rhBMP-2 (preparation method was described in Embodiment 3) was placed in the lower chamber.

After culturing for 24 hours, the Transwell membrane was first scraped with a cotton swab on the upper surface to remove adherent cells, and then separated from the insert.

The cells that migrated to the lower side of the membrane were fixed with 4% paraformaldehyde for 30 minutes and stained with 0.1% crystal violet for 8 minutes.

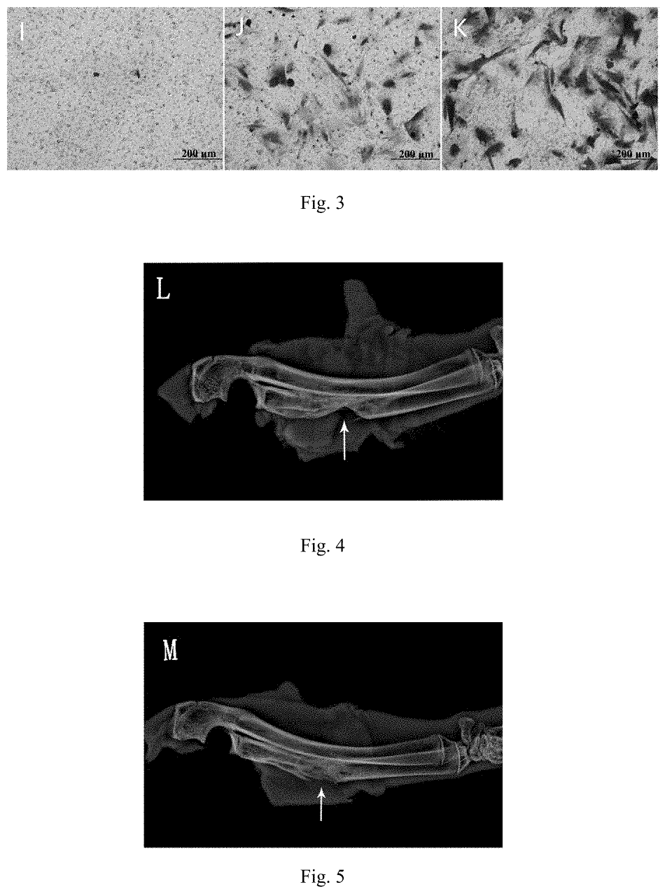

The cell growth situation on the lower surface of the membrane was observed using a 200×microscope. FIG. 3 were crystal violet staining images of an in vitro cell migration experiment, in which the dark part represented the bone marrow mesenchymal stem cells. I was a blank control group, J was a bone matrix group not loaded with the cytokine (preparation method was described in Embodiment 1), and K was a bone scaffold group loaded with the cytokine rhBMP-2 (preparation method was described in Embodiment 3). As can be seen from the comparison between the control group and the group not loaded with the cytokine, the bone scaffold loaded with the cytokine rhBMP-2 according to the embodiments of the present application had an obvious cell migration effect. The present embodiment showed that the bone scaffold loaded with the cytokine via supercritical carbon dioxide had a good effect of promoting the migration of the bone marrow mesenchymal stem cells.

### Embodiment 5

1) A 3-4 week old New Zealand white rabbit was placed on an animal experiment table in ventral decubitus, with four limbs abducted and fixed on two sides of the table.

2) The skin of the rabbit's two forelimbs was prepared and sterilized, and a sterile drape was spread; an incision about 2 cm in length was made in the middle of the radius of each forelimb, the skin, the subcutaneous tissue and deep fascia were cut open and the muscle was separated layer by layer, and the periosteum was peeled off by use of a periosteal detacher; a portion of the rabbit's each radius about 1.2 cm to 1.5 cm in length was cut out by a wire saw, and the bone defects were generated.

3) The bone matrix prepared in the Embodiment 1 and the bone scaffold loaded with the cytokine rhBMP-2 prepared in the Embodiment 3 were implanted respectively, the periosteum was sutured, suture was performed until to the skin layer by layer, and after the surgery, antibiotics were injected intramuscularly for 3 days.

FIG. 4 was an X-ray film of the supercritically cleaned and sterilized bone matrix (preparation method was described in the Embodiment 1) taken after implanted in the bone defect area of the rabbit radius for 1 month, and FIG. 5 was an X-ray film of the bone scaffold loaded with the cytokine rhBMP-2 (preparation method was described in the Embodiment 3) taken after implanted in the bone defect area of the rabbit radius for 1 month. As can be seen from analysis of the X-ray films, there were fusiform callus shadows around the bone defects in both FIG. 4 and FIG. 5, and obvious bone callus was formed after the implantation of the bone scaffold loaded with the cytokine rhBMP-2, and more callus was formed. The present embodiment showed that tissue engineering bone scaffold loaded with the cytokine well promoted the regeneration and repair of the bone defect.

The above is only the specific implementation of the present application, hut the protection scope of the present application is not limited to this. Any person skilled in the art can easily think of various equivalent modifications or replacements within the technical scope disclosed by the present application, and these modifications or replacements should fall within the protection scope of the present application. Therefore, the protection scope of the present application shall be limited by the protection scope of the claims.

1. A method for preparing tissue engineering bone scaffold, the method comprising the following steps:
a cleaning step, subjecting tissue engineering bone material to supercritical fluid cleaning treatment to remove soft tissue in the bone material and obtain an initial bone matrix, wherein the bone material is one or more of allogeneic cancellous bone, xenogeneic cancellous bone and autologous cancellous bone;
a sterilization step, sterilizing the initial bone matrix by supercritical fluid to obtain a bone matrix; and
a compounding step, placing the bone matrix and a cytokine in a supercritical fluid environment, wherein the cytokine is carried into pores of the bone matrix by the supercritical fluid and compounded with the bone matrix to obtain the bone scaffold.

2. The method according to claim 1, wherein the cleaning step comprises:
placing the bone material in a supercritical fluid environment to dissolve the soft tissue into the supercritical fluid and thus remove the soft tissue, wherein a pressure in the supercritical fluid environment is 9 MPa to 15 MPa, and a temperature of the supercritical fluid environment is 37° C. to 45° C.

3. The method according to claim 2, wherein in the cleaning step:
the supercritical fluid contains a first additive, and the first additive is hydrogen peroxide solution, wherein a mass percentage content of hydrogen peroxide in the hydrogen peroxide solution is 1% to 5%.

4. The method according to claim 3, wherein a volume ratio of the hydrogen peroxide solution to the supercritical fluid is 1:1000 to 1:2000.

5. The method according to claim 3, further comprising a preliminary cleaning step before the cleaning step:
subjecting the bone material to at least one of ultrasonic centrifugal cleaning and high-pressure water gun washing by use of a washing liquid, and the washing liquid is one or more of water, alcohols and ketones.

6. The method according to claim 5, further comprising a pretreatment step before the preliminary cleaning step:
mechanically clearing the bone material to remove the soft tissue on a surface of the bone material;
cleaning bone pieces with phosphate buffered saline solution;
freezing the cleaned bone material at −10° C. to −50° C. for 10 h to 48 h, and then freezing the bone material at −50° C. to −100° C. for 10 h to 48 h.

7. The method according to claim 6, further comprising the following step before cleaning the bone pieces with phosphate buffered saline solution:
cutting the mechanically cleared bone material into bone pieces having a predetermined volume.

8. The method according to claim 1, wherein the sterilization step comprises:
making the supercritical fluid to penetrate into the initial bone matrix to sterilize the initial bone matrix, wherein a pressure of the sterilization is 12 MPa to 15 MPa, and a temperature of the sterilization is 37° C.

9. The method according to claim 8, wherein in the sterilization step, the supercritical fluid contains a second additive, and the second additive is one or more of peroxyacetic acid solution and hydrogen peroxide solution;
a mass percentage content of peroxyacetic acid in the peroxyacetic acid solution is 5% to 20%; and
a mass percentage content of the hydrogen peroxide in the hydrogen peroxide solution is 1% to 5%.

10. The method according to claim 9, wherein the second additive is a mixed solution of the peroxyacetic acid solution and the hydrogen peroxide solution, and a volume ratio of the peroxyacetic acid solution and the hydrogen peroxide solution in the mixed solution is 1:9 to 9:1.

11. The method according to claim 10, wherein a volume ratio of the peroxyacetic acid solution, the hydrogen peroxide solution and the supercritical fluid is 3 to 4:1:70000 to 100000.

12. The method according to claim 1,
wherein a pressure of the supercritical fluid environment is 8 MPa to 12 MPa, and a temperature of the supercritical fluid environment is 37° C. to 45° C.

13. The method according to claim 1, wherein the compounding step comprises:
loading the cytokine on the bone matrix in a sterile environment with a temperature lower than 25° C. to obtain a mixture;
placing the mixture in the supercritical fluid, wherein the cytokine is carried into the pores of the bone matrix by the supercritical fluid and compounded with the bone matrix to obtain the bone scaffold.

14. The method according to claim 1, comprising at least one of the following features:
in the cleaning step, the sterilization step and the compounding step, the supercritical fluids are independently selected from one or more of supercritical carbon dioxide, supercritical water, supercritical alcohol and supercritical alkane; and
in the compounding step, the cytokine is one or more of rhBMP-2, TGF-β family and VEGF.

15. The method according to claim 1, further comprising a washing step after the sterilization step:
washing the sterilized initial bone matrix with phosphate buffered saline solution and drying the washed initial bone matrix.

16. A tissue engineering bone scaffold prepared by the method according to claim 1, wherein the bone scaffold comprises a bone matrix with a porous structure and a cytokine loaded into the pores of the bone matrix.

17. The method according to claim 2, wherein the temperature of the supercritical fluid environment is 38° C. to 42° C.

18. The method according to claim 3, wherein the mass percentage content of hydrogen peroxide in the hydrogen peroxide solution is 3%.

19. The method according to claim 4, wherein the volume ratio of the hydrogen peroxide solution to the supercritical fluid is 1:1250.

20. The method according to claim 8, wherein the temperature of the sterilization is 38° C. to 42° C.

21. The method according to claim 9, wherein the mass percentage content of peroxyacetic acid in the peroxyacetic acid solution is 18%, and the mass percentage content of the hydrogen peroxide in the hydrogen peroxide solution is 3%.

22. The method according to claim 10, wherein the volume ratio of the peroxyacetic acid solution and the hydrogen peroxide solution in the mixed solution is 2:1 to 6:1.

23. The method according to claim 12, wherein the pressure of the supercritical fluid environment is 9.9 MPa, and the temperature of the supercritical fluid environment is 38° C. to 42° C.
